(19) United States
(12) Patent Application Publication
(10) Pub. No.: US 2026/0315523 A1
(43) Pub. Date: Oct. 15, 2026

(54) Topical Compositions and Delivery Systems to Decrease Attraction of Insects and Mosquitoes via Modifying Skin Chemical Emissions

(71) Applicant: Nisreen Soufi, North Hollywood, CA (US)

(72) Inventor: Nisreen Soufi, North Hollywood, CA (US)

(73) Assignee: Naturemomo Inc., North Hollywood, CA (US)

(21) Appl. No.: 19/369,136

(22) Filed: Oct. 24, 2025

Related U.S. Application Data
(60) Provisional application No. 63/711,695, filed on Oct. 24, 2024.

(51) Int. Cl. A61K 33/06; A61K 9/00; A61K 33/24; A61P 33/14

(52) U.S. Cl. CPC A61K 33/06; A61K 9/0014; A61K 33/24; A61P 33/14

(57) ABSTRACT

A topical composition and method are described for reducing attraction of host-seeking insects to human skin. The composition includes a magnesium-based or aluminum-based mineral salt in a cosmetically acceptable carrier and is applied to skin to alter skin emissions, including volatile organic compounds and metabolic byproducts associated with mosquito host-seeking behavior. The compositions may be formulated as sprays, balms, sticks, creams, gels, roll-ons, lotions, emulsions, or wipes. By modifying skin-associated chemical cues rather than merely masking odor with volatile fragrances, the compositions reduce insect attraction and may provide a non-toxic, long-lasting personal-care approach for decreasing mosquito bites and other insect interactions.

### CROSS-REFERENCE TO RELATED APPLICATIONS

This application claims priority to U.S. Provisional Patent Application No. 63/711,695, filed Oct. 24, 2024, the entire contents of which are incorporated herein by reference.

### BACKGROUND OF THE INVENTION

Mosquitoes and other biting insects use volatile organic compounds (VOCs) and metabolic byproducts emitted from human skin as primary cues to locate their hosts. Skin emissions such as lactic acid, ammonia, and short-chain fatty acids, often produced or modified by skin microbiota, are widely recognized for their central role in human attractiveness to mosquitoes (Verhulst et al., 2016; de Lacy Costello et al., 2014; Smallegange et al., 2005). This underlies the public health and consumer need for improved ways to reduce mosquito-borne diseases and the discomfort caused by insect bites.

Traditional repellents such as N,N-diethyl-meta-toluamide (DEET) function as true deterrents by interacting with specific insect odorant receptors and thereby preventing host detection and biting (Syed and Leal, 2008; Leal, 2014). In contrast, commonly used essential oils like citronella and lemon eucalyptus are volatile masking agents; they obscure human emissions and provide only short-lived or partial protection due to rapid volatility and limited efficacy (Maia and Moore, 2011; Fradin and Day, 2002).

Mineral salts, particularly magnesium and aluminum salts, have long been used as actives in deodorant and antiperspirant products. These compounds alter skin pH and influence sweat and microflora composition, thereby inhibiting growth of odor-causing bacteria and modifying the nature and amount of VOCs emitted from skin (Troccaz et al., 2009; Discenza, 2022; Strachan et al., 2018).

Recent research (Verhulst et al., 2016) has demonstrated that certain volatile organic compounds (VOCs) from human skin can influence mosquito attraction, and tested ester deodorant compounds, such as isopropyl tetradecanoate, in vitro for their effects on mosquito response. However, the topical use of mineral salts, specifically magnesium and aluminum-based compounds recognized for their prominent role in controlling body odor, was not disclosed, suggested, or tested as an approach for modulating skin emissions to deter insects. Accordingly, the present invention addresses a novel and unmet need for topical compositions that utilize these mineral salts to effectively reduce insect attraction and bites.

### REFERENCES

Verhulst N O, Bakker J W, Fries E H, Beijleveld H, Smallegange R C, Takken W. Attractiveness of volatiles from different body parts to the malaria mosquito Anopheles gambiae. Scientific Reports. 2016; 6:27141.
de Lacy Costello B, Amann A, Al-Kateb H, et al. A review of the volatiles from the healthy human body. Journal of Breath Research. 2014; 8 (1):014001.
Smallegange R C, Qiu Y T, van Loon J J, Takken W. Synergism between ammonia, lactic acid and carboxylic acids as kairomones in host-seeking behaviour of the malaria mosquito. Chemical Senses. 2005; 30 (2):145-152.
Syed Z, Leal W S. Mosquitoes smell and avoid the insect repellent DEET. Proceedings of the National Academy of Sciences. 2008; 105 (36):13598-13603.
Leal W S. The enigmatic reception of DEET-the gold standard of insect repellents. Current Opinion in Insect Science. 2014; 6:93-98.
Maia M F, Moore S J. Plant-based insect repellents: a review of their efficacy, development and testing. Malaria Journal. 2011; 10 (Suppl 1):S11.
Fradin M S, Day J F. Comparative efficacy of insect repellents against mosquito bites. New England Journal of Medicine. 2002; 347 (1):13-18.
Troccaz M, Starkenmann C, Niclass Y, van de Waal M, Clark A J. Full body odor profile: gender, age, menstrual cycle and individual factors. Chemical Senses. 2009; 34 (7):493-503.
Discenza D. The effect of habitual and experimental antiperspirant and deodorant use on the human axillary microbiome. PeerJ. 2022; 10:e13611.
Strachan C R, Skalina K A, Bowdish D M, Embleton M L, Yost C K. Axillary microbiota: quantitative culture and application in two cohort studies. Applied and Environmental Microbiology. 2018; 84(7):e02128-17.

### BRIEF SUMMARY OF THE INVENTION

This invention discloses topical compositions containing magnesium-based or aluminum-based mineral salts individually or in selected embodiments, formulated for skin application to modulate skin VOC or metabolic byproduct emissions and thereby reduce insect attraction. These compositions operate by altering the skin microenvironment, changing pH, sweat-related chemistry, and microbiota-associated metabolites, thereby reducing the release or detectability of known mosquito-attracting compounds including lactic acid, ammonia, and short-chain fatty acids. Unlike volatile masking agents or neurotoxic repellents, the innovation targets the underlying source of chemical signals used by insects.

The compositions can be delivered as sprays, balms, sticks, creams, emulsions, gels, roll-ons, or wipes. Sprays with high skin coverage are preferred for exposed areas, producing a visible or partially visible mineral layer for application confirmation. Example working formulations were observed to reduce mosquito bites comparably or better than commercial plant-oil-based repellents.

### BRIEF DESCRIPTION OF THE DRAWINGS

No drawings are included. The invention can be understood from the written description without drawings.

### DETAILED DESCRIPTION OF THE INVENTION

### Definitions

Magnesium compounds include magnesium hydroxide, magnesium carbonate, magnesium oxide, magnesium sulfate, magnesium chloride, magnesium stearate, and magnesium lactate.

Aluminum compounds include aluminum chlorohydrate, aluminum zirconium tetrachlorohydrex gly, aluminum zirconium trichlorohydrex gly, aluminum sesquichlorohydrate, aluminum hydrochloride, aluminum chloride, aluminum sulfate, and aluminum starch octenylsuccinate.

Cosmetically acceptable carrier includes any safe formulation for topical skin application, including water, ethanol, propylene glycol, glycerin, emollients, film formers, stabilizers, preservatives, oils, waxes, and silicone-based carriers.

Insect means any biting or host-seeking arthropod attracted by human skin emissions, including mosquitoes, biting flies, gnats, midges, fleas, ticks, and other arthropods that use skin-associated chemical cues.

### Compositional Variants

Active Ingredient: Magnesium-based or aluminum-based compound from the above lists, optionally formulated so that a primary active mineral salt is used without DEET, citronella oil, lemon eucalyptus oil, pyrethrins, or p-menthane-3,8-diol.

Carriers and Additives: Film-forming agents, solvents including water, ethanol, and propylene glycol, humectants including glycerin, emollients, stabilizers, preservatives, waxes, and viscosity modifiers.

### Formulation Examples

### Example 1: Magnesium Hydroxide Spray

Magnesium hydroxide: 5% w/w
Water: 40% w/w
Ethanol: 30% w/w
Propylene glycol: 15% w/w
PVP/VA copolymer: 2% w/w
Dimethicone: 2% w/w
Preservative: 0.5% w/w
Adjust pH to 8.0

### Example 2: Magnesium Stearate Balm

Magnesium stearate: 4% w/w
Cyclopentasiloxane: 35% w/w
Stearyl alcohol: 20% w/w
Dimethicone: 8% w/w
Wax base: 20% w/w
Castor oil: 10% w/w
Preservative: 0.5% w/w
pH 7.0-8.0

### Example 3: Magnesium Chloride Cream

Magnesium chloride: 3% w/w
Water: 65% w/w
Glycerin: 8% w/w
Caprylic/capric triglyceride: 10% w/w
Cetearyl alcohol: 7% w/w
Dimethicone: 4% w/w
PEG-8 distearate: 2% w/w
Preservative: 0.5% w/w
pH 6.0-7.5

### Example 4: Aluminum Chlorohydrate Spray

Aluminum chlorohydrate: 8% w/w
Water: 60% w/w
Ethanol: 20% w/w
Propylene glycol: 8% w/w
Cetearyl alcohol: 3% w/w
PEG-8 distearate: 0.9% w/w
Preservative: 0.5% w/w
pH 5.0-5.5

### Example 5: Aluminum Zirconium Tetrachlorohydrex Gly Stick

Aluminum zirconium tetrachlorohydrex gly: 12% w/w
Cyclopentasiloxane: 45% w/w
Stearyl alcohol: 18% w/w
Dimethicone: 7% w/w
Wax base: 17% w/w
Castor oil: 2% w/w
Preservative: 0.5% w/w
pH 5.0-5.5

### Preparation and Use Protocol

Dissolve or disperse mineral salt in water and mix well. Add solvents and humectants for uniformity. Add film formers, emollients, and preservatives; use stirrer or high shear mixing. For balms and sticks, add molten wax, pour, and cool to solidify. For sprays, filter and fill into atomizer bottles. Adjust pH as required. Sprays may have viscosity of about 10-100 cP. Balms may have viscosity of about 3000-5000 cP. Shelf life may exceed 12 months in sealed, opaque packaging.

### Use Cases

Children: Magnesium chloride spray prior to outdoor play; monitored bites and irritation. Athletes: Magnesium hydroxide stick pre-exercise; effective against mosquitoes in humid conditions. Sensitive Skin: Cream-based magnesium chloride for eczema-prone users; no irritation and bite reduction observed. Hot/Cold Climates: Magnesium and aluminum formulations tested in tropical and temperate regions.

### Mechanistic Detail

Topical magnesium and aluminum salts modify the skin microbiome, skin pH, sweat-related metabolites, and volatile organic compound emission profile. The resulting change in emitted host-seeking cues decreases mosquito-attracting signals. Studies (Verhulst et al., 2016; Discenza, 2022; Strachan et al., 2018) support the role of skin odorants and microbiota-associated compounds in mosquito attraction and skin odor emission.

### Experimental Evidence

Multiple field trials are described comparing magnesium-based formulations to essential oil repellents. In seven individual uses across multiple users and settings, treated skin exhibited 0-2 bites compared to 6-10 bites for untreated skin or skin treated with essential oils.

### Industrial Notes

The compositions are scalable using standard cosmetic production, UV-resistant packaging, expiration labeling, and regulatory compliance in the United States, Europe, Japan, and other jurisdictions. Ingredients may be cosmetic-grade and products may be stable for 12-24 months.

### INDUSTRIAL APPLICABILITY

These compositions and devices may be used in personal care, public health, outdoor recreation, travel, occupational, or medical contexts where non-toxic, long-lasting, non-volatile minimization of insect attraction is desired. Products are suitable for industrial scale manufacture and broad geographic application.

### REFERENCES

Verhulst N O, Bakker J W, Fries E H, Beijleveld H, Smallegange R C, Takken W. Attractiveness of volatiles from different body parts to the malaria mosquito Anopheles gambiae. Scientific Reports. 2016; 6:.
de Lacy Costello B, Amann A, Al-Kateb H, et al. A review of the volatiles from the healthy human body. Journal of Breath Research. 2014; 8 (1):014001.
Smallegange RC, Qiu Y T, van Loon J J, Takken W. Synergism between ammonia, lactic acid and carboxylic acids as kairomones in host-seeking behaviour of the malaria mosquito. Chemical Senses. 2005; 30 (2):145-152.
Syed Z, Leal W S. Mosquitoes smell and avoid the insect repellent DEET. Proceedings of the National Academy of Sciences. 2008; 105 (36):13598-13603.
Leal W S. The enigmatic reception of DEET-the gold standard of insect repellents. Current Opinion in Insect Science. 2014; 6:93-98.
Maia M F, Moore S J. Plant-based insect repellents: a review of their efficacy, development and testing. Malaria Journal. 2011; 10 (Suppl 1):S11.
Fradin M S, Day J F. Comparative efficacy of insect repellents against mosquito bites. New England Journal of Medicine. 2002; 347 (1):13-18.
Troccaz M, Starkenmann C, Niclass Y, van de Waal M, Clark A J. Full body odor profile: gender, age, menstrual cycle and individual factors. Chemical Senses. 2009; 34 (7):493-503.
Discenza D. The effect of habitual and experimental antiperspirant and deodorant use on the human axillary microbiome. PeerJ. 2022; 10:e13611.
Strachan C R, Skalina K A, Bowdish D M, Embleton M L, Yost C K. Axillary microbiota: quantitative culture and application in two cohort studies. Applied and Environmental Microbiology. 2018; 84 (7):e02128-17.

1. A topical composition for reducing attraction of host-seeking insects to human skin, the topical composition comprising a magnesium-based compound selected from the group consisting of magnesium hydroxide, magnesium carbonate, magnesium oxide, magnesium sulfate, magnesium chloride, magnesium stearate, and magnesium lactate, and a cosmetically acceptable carrier, wherein the topical composition is configured for application to human skin in an amount effective to alter at least one skin emission selected from volatile organic compounds, ammonia, lactic acid, and short-chain fatty acids.

2. A topical composition for reducing attraction of host-seeking insects to human skin, the topical composition comprising an aluminum-based compound selected from the group consisting of aluminum chlorohydrate, aluminum zirconium tetrachlorohydrex gly, aluminum zirconium trichlorohydrex gly, aluminum sesquichlorohydrate, aluminum hydrochloride, aluminum chloride, aluminum sulfate, and aluminum starch octenylsuccinate, and a cosmetically acceptable carrier, wherein the topical composition is configured for application to human skin in an amount effective to alter at least one skin emission selected from volatile organic compounds, ammonia, lactic acid, and short-chain fatty acids.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. The topical composition of claim 1, wherein the magnesium-based compound is present in an amount of about 0.5% to about 12% by weight of the topical composition.

15. The topical composition of claim 1, wherein the topical composition has a pH of about 6.0 to about 8.5 after formulation.

16. The topical composition of claim 1, wherein the magnesium-based compound comprises magnesium hydroxide and the cosmetically acceptable carrier comprises water, ethanol, propylene glycol, a film-forming polymer, and a silicone emollient.

17. The topical composition of claim 1, wherein the magnesium-based compound comprises magnesium chloride and the cosmetically acceptable carrier comprises water, glycerin, an emollient oil, a fatty alcohol, and a preservative.

18. The topical composition of claim 1, wherein the topical composition is in the form of a spray, balm, stick, wipe, roll-on, gel, emulsion, cream, or lotion.

19. The topical composition of claim 1, wherein the topical composition is substantially free of DEET, citronella oil, lemon eucalyptus oil, p-menthane-3,8-diol, and pyrethrins.

20. The topical composition of claim 2, wherein the aluminum-based compound is present in an amount of about 0.5% to about 12% by weight of the topical composition.

21. The topical composition of claim 2, wherein the topical composition has a pH of about 4.0 to about 6.5 after formulation.

22. The topical composition of claim 2, wherein the aluminum-based compound comprises aluminum chlorohydrate and the cosmetically acceptable carrier comprises water, ethanol, propylene glycol, a fatty alcohol, an emulsifier, and a preservative.

23. The topical composition of claim 2, wherein the aluminum-based compound comprises aluminum zirconium tetrachlorohydrex gly and the cosmetically acceptable carrier comprises a silicone carrier, stearyl alcohol, dimethicone, a wax base, and an oil.

24. The topical composition of claim 2, wherein the topical composition is in the form of a spray, balm, stick, wipe, roll-on, gel, emulsion, cream, or lotion.

25. The topical composition of claim 2, wherein the topical composition is substantially free of DEET, citronella oil, lemon eucalyptus oil, p-menthane-3,8-diol, and pyrethrins.

26. The topical composition of claim 1, wherein the topical composition is packaged in a spray device configured to dispense droplets having an average droplet diameter of about 50 micrometers to about 200 micrometers.

27. A method of reducing attraction of host-seeking insects to human skin, the method comprising applying to exposed human skin a topical composition comprising a mineral salt active ingredient and a cosmetically acceptable carrier, wherein the mineral salt active ingredient comprises a magnesium-based compound or an aluminum-based compound, and wherein the applying alters a skin emission profile of the exposed human skin.

28. The method of claim 27, wherein the magnesium-based compound is selected from the group consisting of magnesium hydroxide, magnesium carbonate, magnesium oxide, magnesium sulfate, magnesium chloride, magnesium stearate, and magnesium lactate.

29. The method of claim 27, wherein the aluminum-based compound is selected from the group consisting of aluminum chlorohydrate, aluminum zirconium tetrachlorohydrex gly, aluminum zirconium trichlorohydrex gly, aluminum sesquichlorohydrate, aluminum hydrochloride, aluminum chloride, aluminum sulfate, and aluminum starch octenylsuccinate.

30. The method of claim 27, wherein the topical composition is applied to skin at least once before outdoor exposure and forms a visible or partially visible mineral-containing layer on the skin.

31. The method of claim 27, wherein the altered skin emission profile comprises reduced emission or reduced detectability of at least one of lactic acid, ammonia, or a short-chain fatty acid associated with mosquito host-seeking behavior.
